(19) United States
(12) Patent Application Publication
(10) Pub. No.: US 2004/0259182 A1
(43) Pub. Date: Dec. 23, 2004

(54) Arrays for chemiluminescent assays, methods of making the arrays and methods of detecting chemiluminescent emissions on solid supports

(76) Inventors: Brooks Edwards, Cambridge, MA (US); John C. Voyta, Sudbury, MA (US); Robert M. Smith, Stow, MA (US); Steven M. Menchen, Fremont, CA (US)

Correspondence Address: Supervisor, Patent Prosecution Services, PIPER RUDNICK LLP, 1200 Nineteenth Street, N.W., Washington, DC, 20036-2412, US

(21) Appl. No.: 10/462,742

(22) Filed: Jun. 17, 2003

(51) Int. Cl. C12Q 1/42; G01N 21/76

(52) U.S. Cl. 435/21; 422/52; 436/172

(57) ABSTRACT

Arrays modified with chemiluminescent enhancing materials and methods of detecting chemiluminescent emissions on solid supports in the presence of chemiluminescent enhancing materials are described. The arrays include a solid support having a surface layer, a plurality of probes disposed on the surface layer in discrete regions and a chemiluminescent enhancing material. The array may be a high density array. At least some of the probes can be bound either directly or indirectly to an enzyme conjugate comprising an enzyme capable of activating a chemiluminescent substrate. The surface layer can be contacted with a composition comprising the chemiluminescent substrate in the presence of the chemiluminescent enhancing material and the resulting chemiluminescent emissions can be detected. The probes can be polynucleotide or polypeptide probes.

This application is related to U.S. patent application Ser. No. 10/046,730, filed Jan. 17, 2002, pending, and U.S. patent application Ser. No. 10/050,188, filed Jan. 14, 2002, pending (published as U.S. Patent Application Publication No. US 2002/0110828 A1 on Aug. 15, 2002). Each of these applications is incorporated herein by reference in its entirety.

### BACKGROUND

1. Technical Field

The subject matter of this application relates generally to biological assays conducted on a solid phase. More specifically, the subject matter of this application relates to arrays modified with chemiluminescent enhancing materials, methods of making the arrays and methods of detecting chemiluminescent emissions on solid supports.

2. Background of the Technology

Microarray technology provides a useful tool for conducting biological assays. A microarray comprises a large number of different probes each of which are immobilized in different discrete areas on a substrate. For nucleic acid assays, the probes can be nucleic acid or oligonucleotide probes. When a sample is contacted with the microarray, molecules in the sample (i.e., target molecules) can hybridize to probes having complementary or substantially complementary sequences. Detection of the position of the hybridized target molecule on the array (e.g., by detecting a label on the target molecule) indicates the presence of a particular sub-sequence in the sample. Due to the large number of different probes present in a microarray, biological assays on microarrays can be conducted in a massively parallel fashion. Microarrays have therefore proven extremely useful in screening, profiling, and sequencing nucleic acid samples.

Assays conducted on microarrays typically employ fluorescently labeled targets. Fluorescent labels can provide high spatial resolution since the signal is generated by a species (i.e., the fluorescer) which is attached to the support either directly or through a probe-target interaction and which is therefore not free to migrate during the assay. In contrast to fluorophore-labeled targets, the use of enzyme labeled targets and chemiluminescent substrates results in a signaling species (i.e., the activated substrate) which is not attached to the support and which is therefore free to migrate during the assay. Migration of the chemiluminescent species during the assay can reduce the spatial resolution of the assay and can result in inaccurate assay data. As a result, chemiluminescent detection of enzyme labeled targets on microarrays has not been widely employed.

It would be desirable to develop improved methods for the use of chemiluminescent detection in high density formats such as microarrays.

### SUMMARY

According to a first embodiment of the invention, an array for chemiluminescent assays is provided. The array includes a solid support having a surface layer, a plurality of different probes disposed on the surface layer in a plurality of discrete areas, and a chemiluminescent enhancing material. According to this embodiment of the invention, the density of discrete areas on the surface layer is at least 50 discrete areas per cm².

According to a second embodiment of the invention, a method of making an array for chemiluminescent assays is also provided. The method includes spotting probes on a surface layer of a solid support in a plurality of discrete areas and coating the surface layer with a chemiluminescent enhancing material. The surface layer of the solid support can be coated with the chemiluminescent enhancing material either before, during, or after the probes are spotted thereon.

According to this embodiment of the invention, the density of discrete areas on the surface layer is at least 50 discrete areas per cm².

According to a third embodiment of the invention, a method of detecting chemiluminescent emissions on a solid support is provided. The method includes: contacting a surface layer of the solid support with a composition comprising a chemiluminescent substrate capable of being cleaved by an enzyme to produce chemiluminescence and detecting chemiluminescent emissions from the surface layer of the solid support. The composition comprising the chemiluminescent substrate is contacted with the surface layer in the presence of a chemiluminescent enhancing material. A plurality of probes are disposed in a plurality of discrete areas on the surface of the solid support such that the density of discrete areas on the surface layer is at least 50 cm⁻². At least some of the probes are bound to an enzyme conjugate comprising an enzyme capable of activating the chemiluminescent substrate. The method according to this embodiment of the invention can further include contacting the surface layer of the solid support with a sample comprising labeled molecules and incubating the sample on the solid support to allow labeled target molecules in the sample to bind to the solid support.

According to a fourth embodiment of the invention, a composition comprising a chemiluminescent enhancing material and a chemiluminescent substrate is provided. The chemiluminescent enhancing material can be a naturally-occurring macromolecular substance, a synthetic macromolecular substance, or mixtures thereof. The chemiluminescent substrate can be a 1,2-dioxetane moiety.

### BRIEF DESCRIPTION OF THE DRAWINGS

FIG. 1 is a flow chart showing a method of making and using an array according to one embodiment of the invention.

FIGS. 2A-2E are CCD images of microarrays wherein FIG. 2A is an image of a control array having no chemiluminescent enhancing material and FIGS. 2B-2E are images of microarrays having varying concentrations of a chemiluminescent enhancing material coated thereon.

### DETAILED DESCRIPTION

According to an embodiment of the present invention, chemiluminescent enhancing materials are used to improve chemiluminescent detection on a solid support, particularly in microarray applications where highly resolved and high intensity images or signal are required. The chemiluminescent enhancing material can be used to overcoat the surface of the array prior to substrate addition. The present inventors have discovered that coating a solid support with a chemiluminescent enhancing material results in a high intensity chemiluminescent signal at each spot, with excellent spot resolution on high density arrays. When used in arrays (e.g., microarrays), the chemiluminescent enhancing material both enhances the signal to give high intensity spots and reduces the extent of substrate diffusion to improve the resolution of the image.

The use of macromolecular chemiluminescent enhancing materials (particularly when the chemiluminescent enhancing materials are adsorbed to the array surface) has been found by the present inventors to restrict diffusion of an enzymatically deprotected dioxetane and to provide an environment favorable to high quantum yield, spatially resolved chemiluminescent emissions, particularly on high density microarrays.

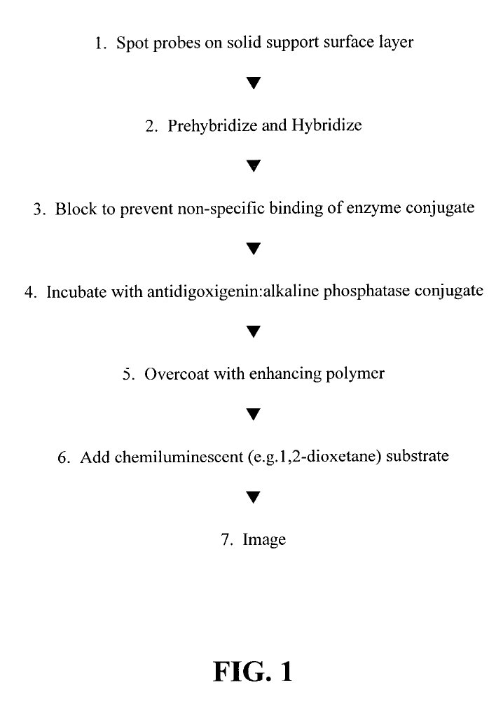

A method of making and using an array according to one embodiment of the invention is shown in FIG. 1. In the process illustrated in FIG. 1, step 1 represents array manufacture whereas steps 2-7 represent steps involved in conducting an assay using the array.

The method illustrated in FIG. 1 is described below. First, the probes are spotted onto a surface layer of a solid support (Step 1). An exemplary material for the surface layer of the solid support is nylon. According to one embodiment, the surface layer can be used alone as the solid support. Alternatively, the surface layer can be disposed on one or more base layers to formt he solid support. Any rigid or semi-rigid material can be used for a base layer. An exemplary base layer material is glass.

After the probes are spotted onto the surface layer of the solid support, the surface layer can be contacted with a sample. In the case of an assay for nucleic acids, the probes on the support surface can be oligonucleotide probes that can hybridize to nucleic acids containing complementary sub-sequences in a sample. As shown in FIG. 1, the support can be pre-hybridized (e.g., to reduce non-specific binding) and then hybridized with the sample (Step 2).

Although oligonucleotide probes are discussed above, any type of probe that is capable of recognizing and binding to a target molecule in the sample can be used. Exemplary probes for nucleic acid targets include, but are not limited to, oligonucleotide probes and cDNA probes. For nucleic acid hybridization assays, the probe comprises a material that is capable of hybridizing with the target nucleic acid. Exemplary probes for protein or polypeptide targets include, but are not limited to, polypeptide probes, aptamer probes, and antibody probes.

The targets in the sample can be labeled with an enzyme capable of cleaving an enzyme labile group on a chemiluminescent substrate. Alternatively, the target can be labeled with a moiety capable of binding with an enzyme conjugate comprising an enzyme capable of cleaving an enzyme labile group on a chemiluminescent substrate. This embodiment, wherein the target is assayed indirectly (i.e., by assaying for an enzyme conjugate which specifically binds to a label on the target molecule) is illustrated in FIG. 1. When the target is assayed indirectly, the target molecules can be labeled with a ligand and an enzyme conjugate capable of binding the ligand can be employed. Exemplary ligand/enzyme conjugate pairs which can be used include, but are not limited to, digoxigenin/antidigoxigenin:enzyme conjugates, biotin/streptavidin:enzyme conjugates, streptavidin/biotin:enzyme conjugates; and fluorescein/antifluorescein:enzyme conjugates.

Alternatively, the target can be unlabeled and detected by hybridization with a second labeled probe that binds to a portion of the target molecule different from that bound by the capture probe on the support surface. The second labeled probe can be labeled directly with an enzyme or with various ligands as set forth above and detected with an enzyme conjugate capable of binding the ligand.

Any enzyme capable of cleaving the chemiluminescent substrate can be used as the enzyme label on the target or in the enzyme conjugate capable of binding the ligand on the target. Exemplary enzymes include, but are not limited to, alkaline phosphatase and beta-galactosidase. As shown in FIG. 1, when the target molecule is a nucleic acid, the target molecule can be labeled with digoxigenin and the enzyme conjugate can be an antidigoxigenin:alkaline phosphatase conjugate.

As shown in FIG. 1, after incubation of the sample on the support surface, the support surface can be blocked to prevent non-specific binding of the enzyme labeled molecule (e.g., an enzyme labeled antibody) to the support surface (Step 3). Afterward, the support surface can be incubated with the enzyme conjugate as shown in Step 4. A chemiluminescent enhancing material can then be coated onto the support surface (Step 5). The chemiluminescent substrate (e.g., a 1,2-dioxetane substrate) can then be contacted with the support surface (Step 6) and chemiluminescent emissions from the support surface can then be detected (Step 7).

Although not shown in FIG. 1, one or more wash steps can be used at numerous points in the process. For example, the support surface can be washed after the sample is contacted with the support surface to remove any unbound labeled molecules.

As shown in FIG. 1, the chemiluminescent enhancing material can be coated onto the surface of the solid support immediately prior to chemiluminescent enzyme substrate addition (i.e., at step 6). Coating with a chemiluminescent enhancing material deposits a layer or web of enhancing material close to the enzyme and provides for localized chemiluminescent enhancement. Further, application of the chemiluminescent enhancing material according to this embodiment of the invention occurs after all of the hybridization and enzyme conjugate addition steps (and any optional washing steps). Therefore, chemiluminescent enhancing material will not be removed from the support surface prior to substrate addition.

Alternatively, the solid support can be coated with chemiluminescent enhancing material at other steps in the process of making or using the array. For example, the chemiluminescent enhancing material may be coated on the solid support at any time from prior to the spotting of the solid support surface with probes to immediately prior to chemiluminescent substrate addition as shown in FIG. 1. The chemiluminescent enhancing material can also be included in the chemiluminescent substrate solution and contacted with the surface of the solid support simultaneously with the substrate.

According to one embodiment of the invention, the coating of the support with the chemiluminescent enhancing material can be performed as part of the array manufacturing process. For example, chemiluminescent enhancing material can be coated onto the solid support surface after spotting the probes (e.g., oligos) on the support surface.

Some chemiluminescent enhancing materials may be more suitable than others for application to the solid support during manufacture of the array. For example, a chemiluminescent enhancing material that is not freely water soluble can be used to reduce the tendency of the chemiluminescent enhancing material to be removed during subsequent processing of the solid support. A chemiluminescent enhancing material which is not freely soluble in water can be applied to the solid support in a suitable solvent or mixture of solvents.

While coating the array immediately prior to chemiluminescent substrate addition is least likely to produce other background artifacts, addition of the chemiluminescent enhancing material at other steps in the assay could also be employed. Specifically, polymer could be applied after contact with the sample (e.g., after hybridization) and before a blocking step (e.g., between steps 2 and 3 as depicted in FIG. 1).

Alternatively, a chemiluminescent enhancing material can be used that acts both as a blocker of non-specific binding and as a chemiluminescent enhancer. The chemiluminescent enhancing material according to this embodiment of the invention can be used in steps 4 or 5 as depicted in FIG. 1. Similarly, the chemiluminescent enhancing material could be applied immediately prior to hybridization (e.g., immediately prior to step 2 in FIG. 1). Alternatively, a chemiluminescent enhancing material can be included in the prehybridization or hybridization buffer for application to the solid support surface (e.g., in step 2 of FIG. 1).

The chemiluminescent enhancing material can also be applied to the support surface in admixture with the chemiluminescent substrate. Accordingly, a composition comprising a chemiluminescent enhancing material and a chemiluminescent substrate is also provided. According to this embodiment of the invention, the polymer may precipitate on or interact with the surface in such a way that no or only limited diffusion of the chemiluminescent product of the enzymatic reaction occurs. A completely water soluble chemiluminescent enhancing material can also be used.

The chemiluminescent enhancing material can be a water-compatible synthetic or naturally-occurring material that can provide a hydrophobic micro-environment of reduced polarity for the light-emitting fragments resulting from the enzymatic cleavage of the chemiluminescent substrate in a polar medium (i.e., a medium consisting of water as a solvent or a mixture of water and other largely or entirely polar substances, such as methanol, acetonitrile, dimethylsulfoxide, dimethyl-formamide and the like). Depending on the precise nature of the micro-environment, the chemiluminescent signal, and/or the chemiluminescent signal to noise ratio can be higher in the presence of the chemiluminescent enhancing material since the chemiluminescent enhancing material can prevent environmental quenching of the chemiluminescent emission from the light-emitting fragments. Additionally, the signal can be more spatially resolved than in the substantially aqueous environment alone since the presence of the chemiluminescent enhancing material can minimize diffusion of the light-emitting fragment resulting from the enzymatic cleavage of the chemiluminescent substrate from the site at which the enzyme reaction occurs.

The chemiluminescent enhancing material can be a macromolecular globular protein having hydrophobic regions. The globular proteins can have molecular weights ranging from about 1,000 to about 800,000 daltons, and preferably from 40,000 to about 100,000 daltons, as determined by SDS gel electrophoresis. Exemplary globular proteins include, but are not limited to mammalian serum albumins such as BSA and HSA and mammalian IgG, IgE, Protein A, and avidins.

The chemiluminescent enhancing material can be a synthetic macromolecular substance (e.g., an oligomeric or polymeric chemiluminescent enhancing material). Exemplary synthetic macrocolecular chemiluminescent enhancing materials include water-soluble or water-miscible solvent soluble polymeric onium salts. A wide variety of polymers of this class have been utilized in the prior art as mordants, or image-receiving layers, in diffusion transfer photographic systems. The onium functionality may be located in the backbone of the polymer (ionenes) or on a group pendant to the backbone. The positively charged, onium functional groups are normally based on nitrogen, phosphorus, or sulfur; however any positively charged grouping may be used. Any of these polymers may be used as macromolecular chemiluminescence enhancing materials. Exemplary of this large class of materials are poly(vinylbenzyl quaternary ammonium salts) having the formula:

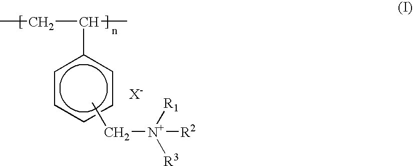

In this formula each group, R¹, R² and R³, each independently represent:

a straight or branched chain unsubstituted alkyl or alkenyl group having from 1 to 20 carbon atoms inclusive (e.g., methyl, ethyl, n-butyl, t-butyl, cetyl, or the like);

a straight or branched chain alkyl group having from 1 to 20 carbon atoms, inclusive, substituted with one or more hydroxy, alkoxy (e.g., methoxy, ethoxy, benzyloxy, or polyethyleneoxy), aryloxy (e.g., phenoxy), amino or substituted amino (e.g., acetamido or cholesteryloxycarbonylamido), or halogen or fluoroalkane or fluoroaryl (e.g., heptafluorobutyl) groups;

an unsubstituted monocycloalkyl group having from 3 to 12 ring carbon atoms inclusive (e.g., cyclohexyl or cyclooctyl);

a substituted monocycloalkyl group having from 3 to 12 ring carbon atoms, inclusive, substituted with one or more alkyl, alkoxy, haloakyl, or fused benzo groups (e.g., dimethylcyclohexyl or tetrahydronaphthyl);

a polycycloalkyl having two or more fused rings, each having from 5 to 12 carbon atoms, inclusive, unsubstituted or substituted with one or more alkyl, alkoxy or aryl groups (e.g., 1-adamantyl or 3-phenyl-1-adamantyl);

an aryl, alkaryl, or aralkyl group having at least one ring and from 6 to 20 carbon atoms in total, unsubstituted or substituted with one or more alkyl, aryl, halogen, fluoroalkyl or fluoroaryl groups (e.g., phenyl, naphthyl, pentafluorophenyl, ethylphenyl, benzyl, chloro- or fluorobenzyl or phenylbenzyl);

At least two of the above R groups, together with the quaternary atom to which they are bonded, can form a saturated or unsaturated, unsubstituted or substituted nitrogen-containing, nitrogen and oxygen-containing, or nitrogen and sulfur-containing ring having from 3 to 5 carbon atoms, inclusive, and 1 to 3 heteroatoms, inclusive, and which may be benzoannulated, e.g., 1-pyridinium, 1-(3-alkyl or aralkyl)imidazolium, morpholinium, alkyl or acylpiperidinium, benzoxazolium, benzothiazolium, or benzimidazolium groups.

The symbol X⁻ represents a counterion, which can include alone, or in combination, moieties such as halide (e.g., chloride or bromide), sulfate, alkylsulfonate (e.g., methanesulfonate), triflate, arylsulfonate (e.g., p-toluenesulfonate), perchlorate, alkanoate (e.g., acetate), arylcarboxylate, or a fluorescent counterion (e.g., fluorescein or fluorescein derivatives), 9, 10-diphenyanthracene sulfonate, or sulforhodamine derivatives.

The symbol n represents a number such that the molecular weight of such poly (vinylbenzyl) quaternary ammonium salts will range from about 8,000 to 1,000,000 or more as determined by the LALLS techniques.

Other exemplary polymeric onium salts which can be used as chemiluminescent enhancing materials include the phosphonium or sulfonium polymers depicted in the following formulae, wherein the definitions for groups, R, X⁻ and n are as given above.

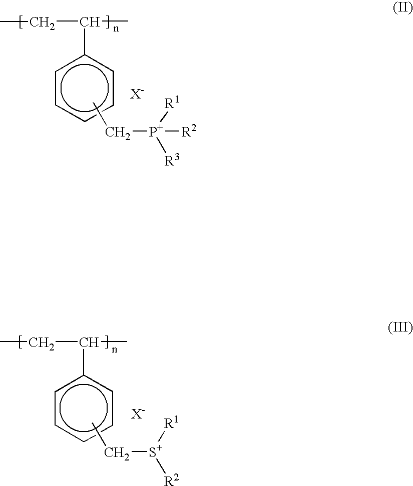

Furthermore, copolymers containing two or more different pendant onium groups may also be used as chemiluminescent enhancing materials. These may be random or block copolymers, which can be synthesized using methods recognized in the art. These copolymers can have the general formula shown below in formula IV or formula V:

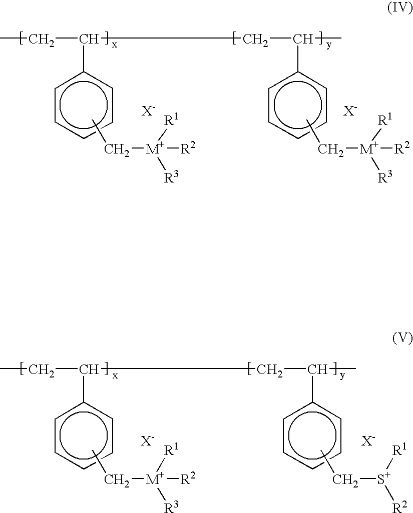

In the above formulae, M may be nitrogen, or phosphorus. Each of the R¹, R² and R³ groups and each X⁻ are as defined above. In formula IV, one or more of the M, R¹, R² or R³ substituents in one of the pendant onium moieties are different than the corresponding substituent in the other pendant onium moiety. The symbols, x and y, represent the mole fraction of the individual monomers comprising the copolymer. The symbols, x and y, may thus individually vary from 0.01 to 0.99, with the sum always equaling one.

Copolymers or block copolymers wherein one of the monomers is an ethylenically unsaturated onium monomer and the other (or others) is charge-neutral can also be used as chemiluminescent enhancing materials. These and other macromolecules capable of providing enhancement of the light emission from chemiluminescent species, such as enzyme-activated 1,2-dioxetanes, can be found in U.S. Pat. Nos. 5,145,772 and 5,827,650. Both of these patents are incorporated herein by reference in their entirety.

Dicationic surfactants can also be using as chemiluminescent enhancing materials. These dicationic surfactants can be represented by the following formula:

X⁻(R₁)₃A⁺CH₂—[LINK]—CH₂ A⁺(R₂)₃ X⁻

wherein:

each A is independently selected from the group consisting of phosphorus and nitrogen atoms;

X is an anionic counterion;

each R₁ and R₂ is independently selected from the group consisting of unsubstituted and substituted alkyl and aralkyl groups containing 1 to 20 carbon atoms such that R₁ and R₂ can be the same or different; and

[LINK] is a carbon chain selected from the group consisting of dialkylenearyl, aryl, alkylene, alkenylene and alkynylene groups containing 4 to 20 carbon atoms. Dicationic surfactants which can be used as chemiluminescent enhancers are described in U.S. Pat. No. 5,451,347, which is herein incorporated by reference in its entirety.

Other water soluble oligomeric, homopolymeric and copolymeric materials can be used as enhancer substances in addition to or instead of the foregoing polymers, including:

poly-N-vinyl oxazolidinones;

polyvinyl carbamates (e.g., polyvinyl propylene carbamate);

polyhydroxyacrylates and methacrylates [e.g., poly(.beta.-hydroxyethyl)methacrylate and polyethyleneglycol monomethacrylates];

amine-containing oligomers (e.g., Jeffamines) quaternized with alkylating or aralkylating agents;

synthetic polypeptides (e.g., polylysine co phenylalanine);

polyvinylalkylethers (e.g., polyvinyl methyl ether);

polyacids and salts thereof [e.g., polyaorylic acids, polymethacrylic acids, polyvinylbenzoic acid, polyethylenesulfonic acid, polyacrylamidomethylpropanesulfonic acid, polymaleic acid and poly(N-vinyl succinamidic acid)];

polyacrylamides and polymethacrylamides derived from ammonia or cyclic and acyclic primary or secondary amines;

polyvinyl alcohol and polyvinyl alcohol copolymers with vinyl acetate, ethylene and the like;

poly 2-, 3- or 4-vinylpyridinium salts where the heterocyclic nitrogen atom

is bonded to a group as defined for R¹, R² and R³ in formula I above;

polyvinylalkylpyrrolidinones (e.g., polyvinylmethyl-pyrrolidinones);

polyvinylalkyloxazolidones (e.g., polyvinylmethyloxazolidones);

branched polyethyleneimines, acylated branched polyethyleneimines, or acylated branched polyethyleneimines further quaternized with alkyl or aralkyl groups;

poly N-vinylamines derived from ammonia or cyclic and acyclic primary or secondary amines, and quaternary salts thereof;

polyvinylpiperidine; or

polyacryloyl, polymethacryloyl or 4-vinylbenzoyl aminimides or polyvinylbenzyl aminimides where the other substituents on the positively charged nitrogen atom may be any of the R¹, R² and R³ groups defined in formula I above.

The above described oligomeric or polymeric chemiluminescent enhancing materials can have molecular weights within the ranges given above for the poly(vinylbenzyl quaternary ammonium salts) of formula I.

Positively charged water-soluble or water-miscible solvent soluble onium monomer salts can also be used as chemiluminescent enhancing materials to enhance chemiluminescent signals on solid supports. The charged monomers can have positively charged onium groups on nitrogen, phosphorus or sulfur, or include any other positively charged grouping in the structure. The counterion can include, either alone or in combination, moieties such as halide (e.g., chloride or bromide), sulfate, alkylsulfonate (e.g., methanesulfonate), triflate, arylsulfonate (e.g., p-toluenesulfonate), perchlorate, alkanoate (e.g., acetate), arylcarboxylate, or a fluorescent counterion.

The chemiluminescent enhancing effect of the polymers described above can be modulated by use of chemiluminescent enhancement additives as described in U.S. Pat. No. 5,547,836, which is hereby incorporated by reference in its entirety. The chemiluminescent enhancement additive can be applied to the solid support surface prior to or after application of the chemiluminescent enhancing material to the surface. Alternatively, the chemiluminescent enhancement additive can be mixed with the chemiluminescent enhancing material and the resulting mixture applied to the solid support surface.

The chemiluminescent enhancement additive can improve the ability of the chemiluminescent enhancing material to form hydrophobic regions in which the dioxetane oxyanion and the resulting emitter can be sequestered, permitting decomposition and chemiluminescence in the absence of water, and therefore, reducing light-quenching reactions caused thereby. The enhancement additives can be drawn from any of a wide variety of compounds. Exemplary enhancement additives include surfactants (e.g., detergents), negatively charged salts and solvents. Surfactants can improve the ability of the chemiluminescent enhancing material to form a hydrophobic region which is relatively stable. The surfactants may be cationic, anionic, zwitterionic or neutral. Another class of enhancement additives which, when added to the solution, appear to improve the ability of the enhancement material to sequester the active dioxetane species, and in any event, lead to further enhancement of the chemiluminescent signal, include negatively charged salts. A third class of enhancement additives also active at very low concentrations are solvents, including alcohols and turpentine.

A fourth effective class of enhancement additives are non-quaternary water-soluble polymers, such as poly(2-ethyl-Z-oxazoline) (PolyOx). While these polymers themselves may induce limited enhancement of the chemiluminescent signal without an increase in background noise, the use of non-quaternary water-soluble polymers in conjunction with polymeric quaternary onium salt enhancement materials can improve the chemiluminescent signal on solid supports such as microarrays.

Further improvements in chemiluminescent signal and S/N can be obtained by independently combining one or more enhancement materials (e.g., globular proteins, synthetic onium or non-onium polymers or copolymers) and one or more enhancement additives.

The chemiluminescent enhancing material can be used to overcoat the solid support surface of the array to provide enhanced, spatially resolved chemiluminescent signals at the surface. Alternatively, macromolecular chemiluminescent enhancing materials can be included in solution with the probes for application to the solid support during spotting or in solution with a chemiluminescent substrate (e.g., a 1,2-dioxetane enzyme substrate) to provide enhanced, spatially resolved chemiluminescent signals.

Exemplary solid supports include those disclosed in U.S. patent application Ser. No. 10/046,730, filed Jan. 17, 2002, pending, which application is incorporated herein by reference in its entirety. For example, the solid support can be any flexible, semi-rigid or rigid surface. The solid support surface may be two-dimensional (i.e., substantially planar), or three-dimensional (i.e., non-planar). For example, the support surface may comprise undulations resulting from stress relaxation of the solid support to increase feature density as set forth in International Publication No. WO 99/53319, and U.S. Patent Application Publication Nos. US 2001/0053497 A1 and US 2001/0053527 A1, which publications are hereby incorporated by reference in their entirety. Exemplary solid support materials include, but are not limited to, silicon, plastic, glass, membrane coated glass, nylon, nitrocellulose, polyethylsulfone, and pigment-impregnated variations thereof. For example, the solid support material can be a supported membrane layer (e.g., membrane coated glass) or an unsupported membrane layer. Exemplary membrane materials include, but are not limited to, nylon, nitrocellulose and polyethersulfone. Exemplary membrane coated glass materials include, but are not limited to, nylon coated glass, nitrocellulose coated glass and polyethersulfone coated glass. Alternatively, the array can be disposed on non-porous surfaces such as glass, silicon dioxide, nylon, or other polymeric materials. The solid support can have any shape. For example, the solid support can be in the form of a planar support (e.g., a glass or membrane coated glass slide) or a non-planar support (e.g., beads).

As set forth above, the probes on the support may be arranged in an array format wherein a plurality of different probes are disposed in discrete areas on the surface of a solid support. The array can be a microarray having a plurality of probes disposed in a discrete area on the surface of a solid support at a relatively high density. The density of the discrete areas in which probes are disposed on the surface layer can, for example, be at least 50 discrete areas per cm², at least 100 discrete areas per cm², at least 400 discrete areas per cm², at least 1,000 discrete areas per cm², at least 25,000 discrete areas per cm², or at least 50,000 discrete areas per cm².

For purposes of determining surface area, the projected (i.e., 2-dimensional) surface area and not the topographical (i.e., 3-dimensional) surface area of the solid support surface is used. The projected and topographical surface areas can differ significantly for solid support surfaces that are not macroscopically planar. For example, an undulated surface will have a topographical surface area that is greater than its projected (i.e., 2-dimensional) surface area. On the other hand, a macroscopically planar surface will have the same projected and topographical surface areas.

The density of a microarray can also be defined by the center to center distance between adjacent spots on the array which is commonly referred to as the “pitch” or the “probe pitch” of the array. Exemplary ranges of probe pitch which can be used include 500 μm or less, 300 μm or less, 250 μm or less, or 80 μm or less. The above ranges are exemplary and other ranges of probe pitch can also be used.

The probes can be spotted on the solid support surface using any known spotting technique. For example, the probes can be deposited onto the solid support surface by a contact method wherein a transfer mechanism used to transfer the probe to the solid support comes into contact with the support surface during deposition. Exemplary transfer mechanisms for contact deposition include a quill pin and a pin-and-ring structure. Alternatively, the probes can be deposited by a non-contact method such as inkjet or piezoelectric printing. The maximum density that can be achieved by spotting may be limited by the particular spotting technique employed.

The probes can also be synthesized on the solid support surface in situ using techniques known in the art. Exemplary in situ manufacturing techniques include, but are not limited to, photolithography and ink jet printing.

A control probe and/or a control label may be positioned in one or more of the same discrete areas on the support surface along with a probe for a target analyte. The signal from the control label can be used to locate features on the array and/or to normalize the signal from the target analyte. Any of the types of controls disclosed in U.S. patent application Ser. No. 10/050,188, filed Jan. 14, 2002, pending, which is incorporated by reference herein in its entirety, may also be used. For example, a control label can be attached to a discrete area on the support surface via attachment of the control label directly to an analyte probe or via attachment to a different molecule attached to the discrete area on the support surface along with the analyte probe. Alternatively, a control label can be attached to a control target capable of binding (e.g., hybridizing) to a control probe attached to one or more discrete areas on the support surface. Any combination or one or more of the above types of controls can be used. For example, a control label and a control probe may both be attached to the support surface and the sample may include a control target (i.e., a target comprising a control label) capable of binding to the control probe.

Any chemiluminescent, enzyme-activatable compound can be used as a chemiluminescent substrate. For example, the chemiluminescent substrate can be a luminol, an acridinium ester, or a 1,2-dioxetane compound. The 1,2-dioxetane compound can be induced to decompose to yield a moiety in an excited state having a heteropolar character that makes it susceptible to environmental effects, particularly to dampening or diminution of luminescence in a polar protic environment. The chemiluminescent compound can be used to determine the presence, concentration or structure of a substance in a polar protic environment, particularly a substance in an aqueous sample.

Among the most effective compounds for this purpose are the stabilized, enzyme-cleavable 1,2-dioxetanes. A number of classes of these chemiluminescent enzyme-triggerable 1,2-dioxetanes, containing a variety of stabilizing functions are known. For example, spiro-bound polycycloalkyl groups either unsubstituted, substituted, or containing sp2 centers are taught in U.S. Pat. Nos. 5,112,960, 5,225,584, and 6,461,876, which are hereby incorporated by reference in their entirety. In addition, branched dialkyl-stabilized, enzyme-triggerable dioxetanes are taught in U.S. Pat. No. 6,284,899, which is also incorporated by reference in its entirety. Substituted furan and pyran-stabilized enzyme-triggerable dioxetanes are taught in U.S. Pat. No. 5,731,445, and European Patent Application Nos. EP 0943618 and EP 1038876, which are also incorporated by reference herein in their entirety. Any of the chemiluminescent substrates disclosed the aforementioned publications can be used.

A dioxetane having a stabilizing moiety can be used as a chemiluminescent substrate. The stabilizing moiety can be chosen based on the requirements of the application. Further, the dioxetanes may also be further substituted with one or more electron withdrawing (e.g. chlorine or fluorine), electron donating (e.g. alkyl or methoxy) groups, or deuterium atoms at any position. This allows tailoring of the quantum yield, emission half-life or pKa [Star dioxetanes] of the enzyme product. The dioxetane can be protected with an enzyme-labile group to form an enzyme cleavable substrate.

As set forth above, stabilized 1,2-dioxetanes (e.g., 1,2-dioxetanes stabilized with an adamantyl group) can be used as the chemiluminescent substrate. This class of dioxetanes can be represented by the following general formula:

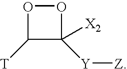

In the above formula, T represents an unsubstituted or substituted cycloalkyl, aryl, polyaryl or heteroatom group (e.g., an unsubstituted cycloalkyl group having from 6 to 12 ring carbon atoms, inclusive); a substituted cycloalkyl group having from 6 to 12 ring carbon atoms, inclusive, and having one or more substituents which can be an alkyl group having from 1 to 7 carbon atoms, inclusive, or a heteroatom group which can be an alkoxy group having from 1 to 12 carbon atoms, inclusive, such as methoxy or ethoxy, a substituted or unsubstituted aryloxy group, such as phenoxy or carboxyphenoxy, or an alkoxyalkyloxy group, such as methoxyethoxy or polyethyleneoxy, or a cycloalkylidene group bonded to the 3-carbon atom of the dioxetane ring through a spiro linkage and having from 6 to 12 carbon atoms, inclusive, or a fused polycycloalkylidene group bonded to the 3-carbon of the dioxetane ring through a spiro linkage and having two or more fused rings, each having from 5 to 12 carbon atoms, inclusive, e.g., an adamant-2-ylidene group.

The symbol Y represents a chromophoric group capable of producing a luminescent substance, which can emit light from an excited energy state upon dioxetane decomposition initiated by enzyme activation.

The symbol X₂ represents hydrogen or an alkyl, aryl, aralkyl, alkaryl, heteroalkyl, heteroaryl, cycloalkyl or cycloheteroalkyl group, e.g., a straight or branched chain alkyl group having from 1 to 7 carbon atoms, inclusive; a straight or branched chain hydroxyalkyl group having from 1 to 7 carbon atoms, inclusive, or an —OR group in which R is a C₁-C₂₀ unbranched or branched, unsubstituted or substituted, saturated or unsaturated alkyl, cycloalkyl, cycloalkenyl, aryl, aralkyl or aralkenyl group, fused ring cycloalkyl, cycloalkenyl, aryl, aralkyl or aralkenyl group, or an N, O or S hetero atom-containing group, or an enzyme-cleavable group containing a bond cleavable by an enzyme to yield an electron-rich moiety bonded to the dioxetane ring. According to one embodiment of the invention, X₂ can be a methoxy group or a trifluoroethoxy group (—CH₂CF₃).

The symbol Z in the above formula represents an enzyme-cleavable group containing a bond cleavable by an enzyme to yield an electron-rich moiety bonded to the dioxetane ring, e.g., a bond which, when cleaved, yields an oxygen anion, a sulfur anion, a nitrogen anion, or an amido anion such as a sulfonamido anion.

An exemplary chemiluminescent substrate is the CDP-Star® substrate (Applied Biosystems, Foster City, Calif.) which is represented by the following chemical formula:

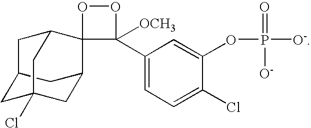

A further exemplary chemiluminescent substrate is the TFE-CDP-Star® substrate (Applied Biosystems, Foster City, Calif.) which is represented by the following chemical formula:

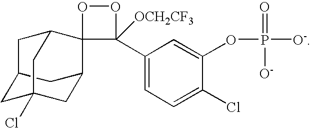

Deuterated dioxetanes can also be used as chemiluminescent substrates. Deuteration of the chemiluminescent dioxetane substrate can result in an increased chemiluminescent signal.

Chemiluminescent substrates other than dioxetanes can also be used. Exemplary chemiluminescent substrates include, but are not limited to, acridan and luminol substrates. When an acridan or luminol substrate is employed, the target molecules can be labeled with an oxidative enzyme such as a peroxidase (e.g., horseradish peroxidase), a catalase or a xanthine oxidase. Acridan substrates for alkaline phosphatase can also be used.

In order to demonstrate the effect of overcoating a high density array with a polymeric onium enhancer, the following experiment was performed. High density arrays were spotted onto an acrylate/azlactone surface. Acrylate/azlactone materials are described in U.S. Pat. No. 6,482,638. The spots on the arrays were spaced approximately 100 microns center to center. The arrays were hybridized with a digoxigenin labeled cDNA sample prepared from liver polyA-mRNA (Stratagene, 0018) using a reverse transcriptase protocol (incorporated digoxigenin labeled dUTP, Enzo, 85996628). The arrays were hybridized overnight and subsequently processed for chemiluminescence detection.

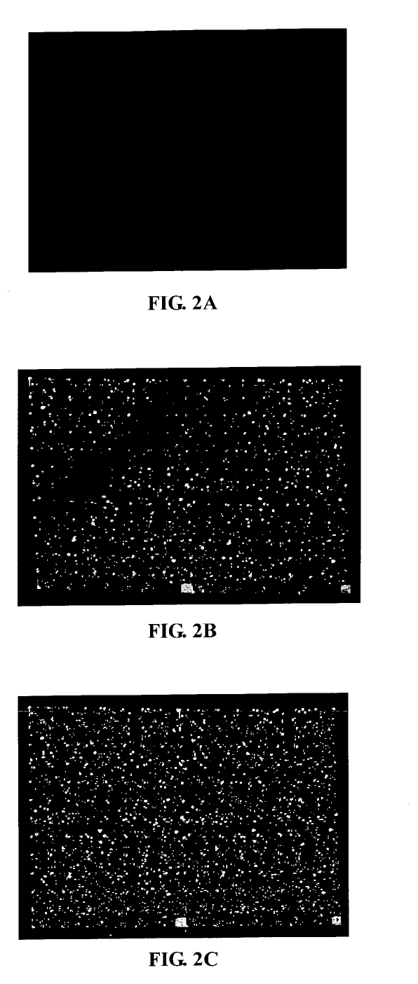

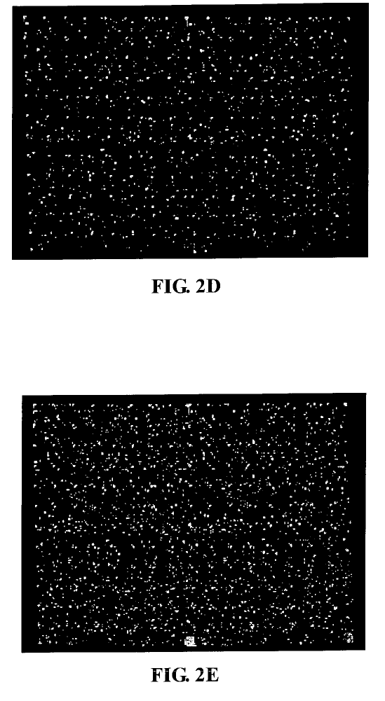

After blocking to prevent nonspecific binding of enzyme labeled reagents, incubation with alkaline phosphatase labeled anti-digoxigenin and washing, arrays were incubated with either 0 mg/ml (FIG. 2A), 0.008 mg/ml (FIG. 2B), 0.04 mg/ml (FIG. 2C), 0.2 mg/ml (FIG. 2D), or 1.0 mg/ml (FIG. 2E) of TPQ polymer enhancer for 20 minutes prior to the addition of TFE-CDP-Star® substrate (2.5 mM in 0.1 M aminomethylpropanol, 1 mM MgCl₂, pH 9.5).

As shown in FIGS. 2A-2E, there is a substantial difference in chemiluminescent signal between the control (i.e., zero polymer used to overcoat the array) and the arrays overcoated with chemiluminescent enhancing material at any of the concentrations tested. In particular, no chemiluminescent signal is visible in the absence of the TPQ enhancing polymer overcoat (FIG. 2A) whereas significant levels of detectable signal are present at all of the TPQ concentrations tested.

The images shown in FIGS. 2A-2E are 25 second images obtained with a prototype ABI 1700 chip imager. CCD intensities of from 200 to 1000 are displayed.

Although TPQ is specifically disclosed as a chemiluminescent enhancing material above, other chemiluminescent enhancing materials can also be used to enhance chemiluminescent emissions on solid supports.

The foregoing description is by way of example only and is not intended to be limiting. Although specific embodiments have been described herein for purposes of illustration, various modifications to these embodiments can be made without the exercise of inventive faculty. All such modifications are within the spirit and scope of the appended claims.

What is claimed is:

1. An array for chemiluminescent assays comprising:
a solid support comprising a surface layer;
a plurality of different probes disposed on the surface layer in a plurality of discrete areas; and
a chemiluminescent enhancing material disposed on the surface layer;
wherein the density of discrete areas on the surface layer is at least 50 discrete areas per cm².

2. The array of claim 1, wherein the chemiluminescent enhancing material comprises a naturally-occurring macromolecular substance.

3. The array of claim 2, wherein the macromolecular substance is a globular protein comprising hydrophobic regions.

4. The array of claim 3, wherein the globular protein is a mammalian serum albumin.

5. The array of claim 4, wherein the serum albumin is bovine serum albumin (BSA) or human serum albumin (HSA).

6. The array of claim 1, wherein the probes are polynucleotide probes or polypeptide probes.

7. The array of claim 1, wherein the chemiluminescent enhancing material comprises a synthetic macromolecular substance.

8. The array of claim 7, wherein the synthetic macromolecular substance is a polymer comprising an onium functional group.

9. The array of claim 7, wherein the synthetic macromolecular substance is a polymer comprising an onium repeating unit represented by the following general formula I or the following general formula II: 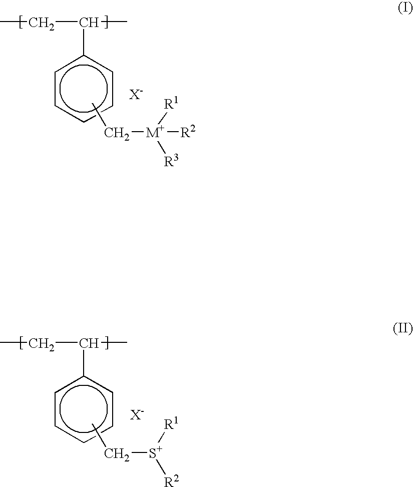
wherein each R₁, R₂ and R₃ independently represent a straight or branched chain alkyl group having from 1 to 20 carbon atoms optionally substituted with one or more hydroxy, alkoxy, aryloxy, amino or substituted amino, amido, ureido, fluoroalkane or fluoroaryl groups; a monocycloalkyl group having from 3 to 12 carbon ring carbon atoms optionally substituted with one or more alkyl, alkoxy or fused benzo groups; a polycycloalkyl group having 2 or more fused rings, each ring having from 5 to 12 carbon atoms optionally substituted with one or more alkyl, alkoxy or aryl groups; an aryl, alkaryl or aralkyl group having at least one ring and from 6 to 20 carbon atoms optionally substituted with one or more alkyl, aryl, fluorine or hydroxy groups; wherein at least two of R₁, R₂ and R₃, together with the quaternary nitrogen atom to which they are bonded, can form a saturated or unsaturated, unsubstituted or substituted nitrogen-containing, nitrogen and oxygen-containing or nitrogen and sulfur-containing ring having from 3 to 5 carbon atoms and 1 to 3 heteroatoms and which may be benzoannulated;
wherein M is a nitrogen or a phosphorus atom; and
wherein X⁻ represents a counter ion.

10. The array of claim 9, wherein the polymer is a copolymer.

11. The array of claim 10, wherein the copolymer comprises two different onium repeating units each of which are represented by the general formula I or the general formula II.

12. The array of claim 1, wherein the density of discrete areas on the surface layer is at least 100 discrete areas per cm².

13. The array of claim 1, wherein the density of discrete areas on the surface layer is at least 1,000 discrete areas per cm².

14. The array of claim 1, wherein the density of discrete areas on the surface layer is at least 25,000 discrete areas per cm².

15. The array of claim 1, wherein the density of discrete areas on the surface layer is at least 50,000 discrete areas per cm².

16. The array of claim 1, wherein the solid support comprises an azlactone functional layer.

17. The array of claim 1, wherein the support surface is non-planar.

18. The array of claim 1, wherein the support surface is planar.

19. The array of claim 1, wherein the solid support comprises a polymer coated glass layer and wherein the support surface comprises the polymer layer.

20. The array of claim 19, wherein the polymer layer is porous.

21. The array of claim 1, further comprising a chemiluminescent enhancement additive.

22. The array of claim 21, wherein the enhancement additive is selected from the group consisting of surfactants, negatively charged salts, alcohols, polyols, poly(2-ethyl-Z-oxazoline), and combinations thereof.

23. The array of claim 21, wherein the enhancement additive is selected from the group consisting of zwitterionic, anionic, cationic and neutral surfactants.

24. The array of claim 1, wherein the chemiluminescent enhancing material is selected from the group consisting of: poly-N-vinyl oxazolidinones; polyvinyl carbamates; polyhydroxyacrylates and methacrylates; amine-containing oligomers quaternized with alkylating or aralkylating agents; synthetic polypeptides; polyvinylalkylethers; polyacids and salts thereof; polyacrylamides and polymethacrylamides derived from ammonia or cyclic and acyclic primary or secondary amines; polyvinyl alcohol and polyvinyl alcohol copolymers; poly 2-, 3- or 4-vinylpyridinium salts; polyvinylalkylpyrrolidinones;
polyvinylalkyloxazolidones; branched polyethyleneimines; acylated branched polyethyleneimines; acylated branched polyethyleneimines further quaternized with alkyl or aralkyl groups; poly N-vinylamines derived from ammonia and quaternary salts thereof; cyclic and acyclic primary or secondary amines and quaternary salts thereof; polyvinylpiperidine; polyacryloyl; polymethacryloyl; 4-vinylbenzoyl aminimides; polyvinylbenzyl aminimides and combinations thereof.

25. A method of making an array for chemiluminescent assays comprising spotting probes on a surface layer of a solid support in a plurality of discrete areas and coating the surface layer with a chemiluminescent enhancing material;
wherein the density of discrete areas on the surface layer is at least 50 discrete areas per cm².

26. The method of claim 25, wherein the solid support is coated with the chemiluminescent enhancing material after spotting probes thereon.

27. The method of claim 25, wherein the solid support is coated with the chemiluminescent enhancing material before spotting probes thereon.

28. A method of detecting chemiluminescent emissions on a solid support, the method comprising:
contacting a surface layer of the solid support with a composition comprising a chemiluminescent substrate capable of being cleaved by an enzyme to produce chemiluminescence; and
detecting chemiluminescent emissions from the surface layer of the solid support;
wherein a plurality of probes are disposed in a plurality of discrete areas on the surface layer, wherein the density of discrete areas on the surface layer is at least 50 discrete areas per cm², wherein at least some of the probes are bound to an enzyme conjugate comprising an enzyme capable of cleaving the chemiluminescent substrate, and wherein the composition comprising the chemiluminescent substrate is contacted with the surface layer in the presence of a chemiluminescent enhancing material.

29. The method of claim 28, further comprising:
contacting the surface layer of a solid support with a sample comprising labeled molecules; and
incubating the sample on the solid support to allow labeled target molecules in the sample to bind to the solid support.

30. The method of claim 28, wherein the chemiluminescent enhancing material comprises a naturally-occurring or synthetic macromolecular substance that can provide a hydrophobic micro-environment for fluorophore containing fragments resulting from the decomposition of a chemiluminescent compound contained in a polar medium.

31. The method of claim 28, wherein the chemiluminescent substrate comprises a 1,2-dioxetane moiety.

32. The method of claim 29, further comprising washing the surface layer between incubating and contacting the surface layer with the chemiluminescent substrate.

33. The method of claim 29, further comprising contacting the surface layer with the chemiluminescent enhancing material.

34. The method of claim 33, wherein the surface layer is contacted with the chemiluminescent enhancing material before the sample is contacted with the surface layer.

35. The method of claim 33, wherein the surface layer is contacted with the chemiluminescent enhancing material after the sample is contacted with the surface layer but before the chemiluminescent substrate is contacted with the surface layer.

36. The method of claim 33, wherein the composition comprising the chemiluminescent substrate further comprises the chemiluminescent enhancing material such that the surface layer is simultaneously contacted with the chemiluminescent enhancing material and the chemiluminescent substrate.

37. The method of claim 33, further comprising spotting the plurality of probes on the surface layer.

38. The method of claim 37, wherein the plurality of probes are spotted on the surface layer and the chemiluminescent enhancing material is contacted with the surface layer simultaneously.

39. The method of claim 37, wherein the surface layer is contacted with the chemiluminescent enhancing material before the sample is contacted with the surface layer.

40. The method of claim 37, wherein the surface layer is contacted with the chemiluminescent enhancing material after the sample is contacted with the surface layer but before the chemiluminescent substrate is contacted with the surface layer.

41. The method of claim 37, wherein the surface layer is simultaneously contacted with the chemiluminescent enhancing material and the chemiluminescent substrate.

42. The method of claim 28, wherein the density of discrete areas on the surface layer is at least 100 discrete areas per cm².

43. The method of claim 28, wherein the density of discrete areas on the surface layer is at least 1,000 discrete areas per cm².

44. The method of claim 28, wherein the density of discrete areas on the surface layer is at least 25,000 discrete areas per cm².

45. The method of claim 28, wherein the density of discrete areas on the surface layer is at least 50,000 discrete areas per cm².

46. The method of claim 28, further comprising contacting the support surface with a composition comprising a blocking agent which inhibits non-specific binding of the enzyme conjugate to the support surface before contacting the surface layer with the composition comprising the enzyme conjugate.

47. The method of claim 29, wherein the target molecules are labeled with digoxigenin and wherein the enzyme conjugate is an antidigoxigenin:enzyme conjugate.

48. The method of claim 28, wherein the enzyme conjugate is an antidigoxigenin:alkaline phosphatase conjugate.

49. The method of claim 28, wherein the enzyme conjugate is bound indirectly to the probe.

50. The method of claim 49, wherein the enzyme conjugate is bound to a target molecule which is bound to the probe.

51. The method of claim 50, wherein the enzyme conjugate comprises an antibody and wherein the target molecule comprises an antigen moiety capable of being bound by the antibody.

52. The method of claim 50, wherein the target molecule is labeled with digoxigenin and wherein the enzyme conjugate is an antidigoxigenin:enzyme conjugate.

53. The method of claim 50, wherein the enzyme conjugate is an antidigoxigenin:alkaline phosphatase conjugate.

54. The method of claim 28, wherein the enzyme conjugate is bound directly to the probe.

55. The method of claim 28, wherein the chemiluminescent substrate is selected from the group consisting of: a 1,2-dioxetane; a luminol; or and acridan.

56. The method of claim 28, wherein the chemiluminescent substrate is a 1,2-dioxetane represented by the following formula: 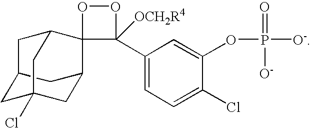
wherein R⁴ is hydrogen or —CF₃.

57. The method of claim 28, wherein the chemiluminescent enhancing material is selected from the group consisting of: a naturally-occurring macromolecular substance; a globular protein comprising hydrophobic regions; a synthetic macromolecular substance; a polymer or copolymer comprising an onium functional group; onium monomers; and combinations thereof.

58. A composition comprising a chemiluminescent enhancing material and a chemiluminescent substrate.

59. The composition of claim 58, wherein the chemiluminescent enhancing material comprises a naturally-occurring macromolecular substance, a synthetic macromolecular substance or mixtures thereof.

60. The composition of claim 58, wherein the chemiluminescent substrate comprises a 1,2-dioxetane moiety.

61. The array of claim 1, wherein the chemiluminescent enhancing material comprises a dicationic material represented by the following formula:
X— (R₁)₃A⁺CH₂—[LINK]—CH₂ A⁺(R₂)₃ X⁻
wherein:
each A is independently selected from the group consisting of phosphorus and nitrogen atoms;
X is an anionic counterion;
each R₁ and R₂ is independently selected from the group consisting of unsubstituted and substituted alkyl and aralkyl groups containing 1 to 20 carbon atoms wherein R_{1 and R}₂ can be the same or different; and
[LINK] is a carbon chain selected from the group consisting of dialkylenearyl, aryl, alkylene, alkenylene and alkynylene groups containing 4 to 20 carbon atoms.
